(19) United States
(12) Patent Application Publication
(10) Pub. No.: US 2009/0018216 A1
(43) Pub. Date: Jan. 15, 2009

(54) POLYMER MICELLE COMPLEX INCLUDING NUCLEIC ACID

(75) Inventors: Kazunori Kataoka, Tokyo (JP); Yuichi Yamasaki, Tokyo (JP); Nobuhiro Nishiyama, Tokyo (JP); Woo-Dong Jang, Tokyo (JP); Anwar Arnida, Tokyo (JP)

Correspondence Address: DARBY & DARBY P.C., P.O. BOX 770, Church Street Station, New York, NY, 10008-0770, US

(73) Assignee: THE UNIVERSITY OF TOKYO, Tokyo (JP)

(21) Appl. No.: 12/281,131

(22) PCT Filed: Sep. 4, 2006
(86) PCT No.: PCT/JP2006/317921

§ 371 Date: Sep. 22, 2008

(30) Foreign Application Priority Data
Mar. 1, 2006 (JP) 2006-054327

(51) Int. Cl. A61K 47/34; C08G 69/48

(52) U.S. Cl. 514/772.3; 525/418

(57) ABSTRACT

It is an object of the present invention to provide: a polyion complex that sufficiently retains a photosensitizing substance in serum and is excellent in terms of structural stability; a nucleic acid polyplex as a constituent thereof; and a device and a kit for delivering a nucleic acid into a cell.

The nucleic acid polyplex of the present invention comprises a cationic polymer represented by general formula (1) and a nucleic acid. The polyion complex of the present invention comprises the nucleic acid polyplex of the present invention and an anionic photosensitizing substance.

### CROSS-REFERENCE TO PRIOR APPLICATION

This is a U.S. National Phase Application under 35 U.S.C. §371 of International Patent Application No. PCT/JP2006/317921 filed Sep. 4, 2006, and claims the benefit of Japanese Patent Application No. 2006-054327, filed Mar. 1, 2006, both of which are incorporated by reference herein. The International Application was published in Japanese on Sep. 7, 2007 as WO 2007/099661 A1 under PCT Article 21(2).

### TECHNICAL FIELD

The present invention relates to a polymer micelle complex that contains a nucleic acid and a photosensitizing substance, a device for delivering a nucleic acid into a cell, and a kit for delivering a nucleic acid into a cell. More specifically, the present invention relates to the aforementioned complex, device, and kit, which can be used for a method for photochemically introducing a nucleic acid into a target cell utilizing photodynamic therapy.

### BACKGROUND ART

Use of a viral vector in gene therapy causes a problem regarding the antigenecity of a viral protein. In order to solve such problem and to realize gene therapy, the development of an effective and safe non-viral vector is extremely important. However, a non-viral vector has been problematic in terms of low gene expression efficiency.

Thus, in recent years, novel non-viral vectors have been developed using various synthetic polymers, and gene expression efficiency has been significantly improved. However, it is extremely difficult for both the non-viral vector and the viral vector to control the site of gene expression in the body. Since a local abnormality of protein expression is observed in many diseases, selective introduction of a gene into a target cell and the expression thereof are extremely important.

Attention has recently been focused on photodynamic therapy (PDT), which is a treatment involving injecting into a body a compound called a “photosensitizer” that reacts with light such as ultraviolet light, visible light, and infrared radiation, and applying such light to a target area so as to treat the target area. This method is a therapy method, wherein the target area, namely, cells in a target tissue, are selectively destroyed as a result of reaction of the photosensitizer compound only in the area to which light has been applied (i.e., the target tissue).

More specifically, in PDT therapy, there is used a photoreactive compound (photosensitizing substance (photosensitizer)), which has high affinity for cells in a target tissue and is efficiently excited by light (e.g. a porphyrin compound). This compound reacts with oxygen molecules in the local environment around the target tissue as a result of being irradiated with light, and it causes photoexcitation of the oxygen molecules, so as to convert the oxygen molecules to singlet oxygen. This singlet oxygen oxidizes peripheral cells and destroys them.

Berg et al. have proposed photochemical internalization (PCI) and a photochemical gene transfection method as means for photoselectively increasing the translocation level of a gene, other nucleic acids and a protein from endosome to cytoplasm (please see K. Berg et al., Cancer Research, 59, 1180-1183 (1999); A. Hogset et al., Human Gene Therapy, 11, 869-880 (2000)). These methods comprise culturing cells in the presence of a commonly used photosensitizing substance, allowing a gene or the like to act on the cells, and then applying light thereto. Thus, in these methods, photodamage is given to the endosomal membrane, and the translocation level of the gene or the like to cytoplasm can be thereby increased.

According to these methods, expression of the function of a gene or the like can be controlled by light irradiation, in principle. However, since photosensitizing substances non-specifically accumulate in cell organelles other than the endosome, significant phototoxicity to a cell as a whole might result. This causes a serious problem to practical application. In reality, Berg et al. have reported that approximately 50% of cells die under conditions necessary for obtaining the maximum gene expression efficiency (please see A. Hogset et al., Human Gene Therapy, 11, 869-880 (2000)).

In order to solve such problem, it has been necessary to develop a novel photosensitizing substance that specifically accumulates in the endosome and selectively results in photodamage to the endosome. Hence, a micelle structure formed by coating a photosensitizing substance with an ionic polymer has been developed. Another micelle structure containing a nucleic acid has also been prepared, separately. Thus, a technique of allowing the two above micelle structures to simultaneously act on a target cell and then delivering the nucleic acid into the cytoplasm has been proposed (please see JP Patent Publication (Kokai) No. 2005-120068 A).

In this method, however, since both micelle structures are different products, it has been difficult for both of them to coexist in all endosomes. Accordingly, the efficiency of introducing a nucleic acid into a target cell has been limited.

Thus, in order to solve the aforementioned difficulty in the coexistence of the two micelle structures in endosomes, a structure (a nucleic acid polyplex) has been produced by binding a “cationic polymer” to a “nucleic acid” acting as a core. Further, an “anionic photosensitizing substance” (e.g., a dendrimer-type substance, etc.) has been allowed to electrostatically interact with the surface of the nucleic acid polyplex structure, so as to form a ternary complex (a polyion complex) (please see N. Nishiyama et al., Nature Materials, 4, 934-941 (2005)).

Nevertheless, in this complex, in the presence of serum, the aforementioned photosensitizing substance tends to be replaced with an anionic protein contained in the serum, and thus the structure of this complex is apt to become unstable. Accordingly, the delivery of this complex via intravenous administration is difficult, and thus this complex is poor in terms of practical application.

### DISCLOSURE OF THE INVENTION

It is an object of the present invention to provide a polyion complex that sufficiently retains a photosensitizing substance in serum and is excellent in terms of structural stability, and has a nucleic acid polyplex as a constituent thereof. It is another object of the present invention to provide a device and a kit for delivering a nucleic acid into a cell.

As a result of intensive studies directed towards achieving the aforementioned objects, the present inventor has found that the aforementioned objects can be achieved by using, as a cationic polymer acting as a constituent of a polyion complex, a specific block copolymer comprising a block portion having a side chain capable of forming a complex with a nucleic acid and a block portion having a side chain capable of forming a complex with an anionic photosensitizing substance, thereby completing the present invention.

That is to say, the present invention includes the following features:

(1) A nucleic acid polyplex, which comprises a cationic polymer represented by the following general formula (1) and a nucleic acid:

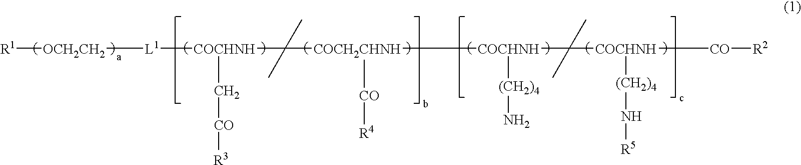

[wherein each of R¹ and R² independently represents a hydrogen atom or a substitutable linear or branched alkyl group containing 1 to 12 carbon atoms;
each of R³ and R⁴ independently represents a residue derived from an amine compound having a primary amine;
R⁵ represents a residue containing a thiol group or a substituent thereof,
L¹ represents NH, CO, or a group represented by the following general formula (5):

—(CH₂)ₚ₁—NH— (5)

(wherein p1 represents an integer between 1 and 5) or the following general formula (6):

-L^{2a}(CH₂)_{q1}-L^{3a}- (6)

(wherein L^{2a} represents OCO, OCONH, NHCO, NHCOO, NHCONH, CONH or COO, L^{3a} represents NH or CO, and q1 represents an integer between 1 and 5);
a represents an integer between 100 and 500; b represents an integer between 5 and 100;
c represents an integer between 20 and 100; and
the symbol “/” indicates that the ratio of the numbers of monomer units described at the left and right sides thereof and the sequence order are arbitrarily determined].

In the nucleic acid polyplex of the present invention, the —R³ group and/or —R⁴ group in the polymer is, for example, a group represented by the following general formula (2):

—[NH—(CH₂)ₘ₁]ₘ₂—X¹ (2)

(wherein X¹ represents a primary, secondary or tertiary amine compound, or an amine compound residue derived from a quaternary ammonium salt; and m1 and m2 are independent from each other and are also independent among the [NH—(CH₂)ₘ₁] units, and m1 represents an integer between 1 and 5 and m2 represents an integer between 1 and 5).

An example of the nucleic acid polyplex of the present invention is a nucleic acid polyplex wherein the —NH₂ group in the polymer and the nucleic acid bind to each other by electrostatic interaction. Another example of the nucleic acid polyplex of the present invention is a nucleic acid polyplex wherein the nucleic acid forms a core portion and the polymer forms a shell portion.

(2) A polyion complex, which comprises the nucleic acid polyplex according to (1) above and an anionic photosensitizing substance.

In the polyion complex of the present invention, an example of the photosensitizing substance is a dendrimer. An example of the dendrimer is a dendrimer having a metalloporphyrin ring.

An example of the polyion complex of the present invention is a polyion complex wherein the —R³ group and/or —R⁴ group in the polymer and the photosensitizing substance bind to each other by electrostatic interaction. Another example of the polyion complex of the present invention is a polyion complex wherein the nucleic acid forms a core portion as a result of being coated with the photosensitizing substance, and the polymer forms a shell portion. A further example of the polyion complex of the present invention is a polyion complex wherein the shell portion comprises a polyethylene glycol chain of the polymer.

(3) A device for delivering a nucleic acid into a cell, which comprises the polyion complex according to (2) above.
(4) A kit for delivering a nucleic acid into a cell, which comprises a cationic polymer represented by general formula (1) (the same as described above) and an anionic photosensitizing substance.
(5) A cationic polymer represented by general formula (1) (as described above).

Moreover, in another aspect, the present invention also provides a polyplex which comprises a cationic polymer represented by general formula (1) (the same as described above) and an anionic substance, and further provides a polyion complex which comprises the aforementioned polyplex and an anionic photosensitizing substance.

### BRIEF DESCRIPTION OF THE DRAWINGS

FIG. 1 is a schematic view of the cationic block copolymer used in the present invention.

FIG. 2 is a schematic view of the nucleic acid polyplex of the present invention.

FIG. 3 is a schematic view of the polyion complex of the present invention, wherein (a) indicates individual constituents and (b) indicates the formed polyion complex.

FIG. 4 is a view showing an absorbance spectrum chart of the nucleic acid polyplex and polyion complex of the present invention.

FIG. 5 is a graph showing the measurement results of the zeta potential of the polyion complex of the present invention.

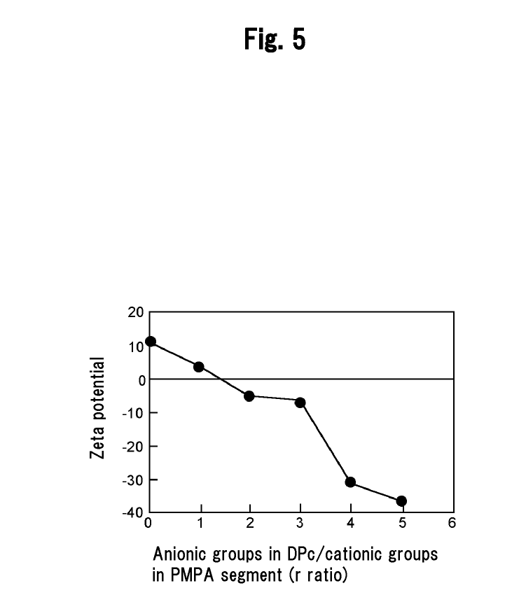

FIG. 6 is a graph showing the relationship between a light irradiation intensity and cytotoxicity in the polyion complex of the present invention.

FIG. 7 is a graph showing the relationship between a light irradiation time and a gene expression level in the polyion complex of the present invention.

FIG. 8 is a graph showing the relationship between a light irradiation time and cytotoxicity in the polyion complex of the present invention.

### NUMERICAL EXPLANATION

1. Cationic block copolymer
2. Block portion having side chain electrostatically binding to nucleic acid
3. Block portion having side chain electrostatically binding to anionic sensitizing substance
4. Block portion of PEG chain
5. Nucleic acid polyplex
6. Nucleic acid
7. Anionic sensitizing substance
8. Polyion complex

### BEST MODE FOR CARRYING OUT THE INVENTION

The present invention will be described in detail below. The examples that follow are provide to illustrate, but not limit, the claimed invention. It will be understood by those skilled in the art that various changes may be made without departing from the scope of the invention and the invention is not to be considered limited in scope to what is shown in the drawings and described in the specification.

The present specification includes all of the contents as disclosed in the specification and/or drawings of Japanese Patent Application No. 2006-054327, which is a priority document of the present application. In addition, all prior art publications, and publications of unexamined applications, patent publications and other patent documents cited herein are incorporated herein by reference in their entirety.

### 1. SUMMARY OF THE PRESENT INVENTION

In order to solve the aforementioned problem regarding the structural instability of the conventional polyion complex, the present inventor considered it necessary to form a complex with an anionic photosensitizing substance, such that the anionic photosensitizing substance cannot easily be substituted with another protein or the like in the presence of serum. For formation of such a complex, the inventor considered it insufficient to laminate an anionic photosensitizing substance on the surface of a nucleic acid polyplex (a nucleic acid and a cationic polymer), as with the conventional technique (coating type), but instead focused on the importance of adding such an anionic photosensitizing substance into such a nucleic acid polyplex (incorporating type). The inventor conducted intensive studies regarding a means for realizing such technique.

As a result, the present inventor developed a polymer having a specific block structure as a cationic polymer used in formation of a nucleic acid polyplex. Thereafter, the inventor succeeded in constructing the aforementioned incorporating-type polyion complex, using the aforementioned polymer.

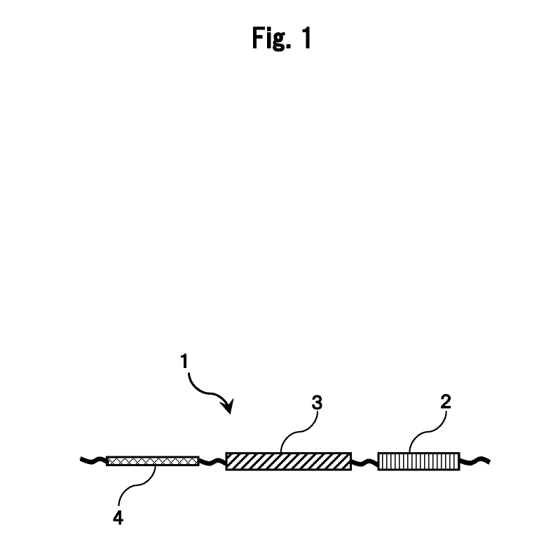

Specifically, as a cationic polymer, a block copolymer 1 as shown in FIG. 1 (a schematic view) was constructed. This polymer 1 comprises a block portion 2 having a side chain electrostatically binding to a nucleic acid (a bond formed by electrostatic interaction) and a block portion 3 having a side chain electrostatically binding to an anionic photosensitizing substance. In addition, a block portion 4 is a block portion consisting of a polyethylene glycol (PEG) chain, and this is an important portion for an increase in bio-compatibility or the like.

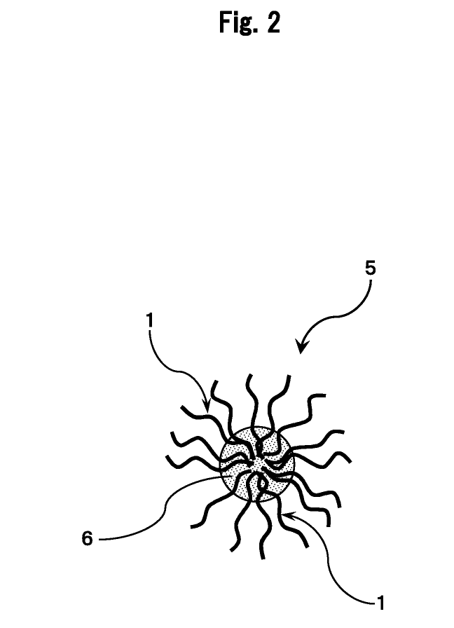

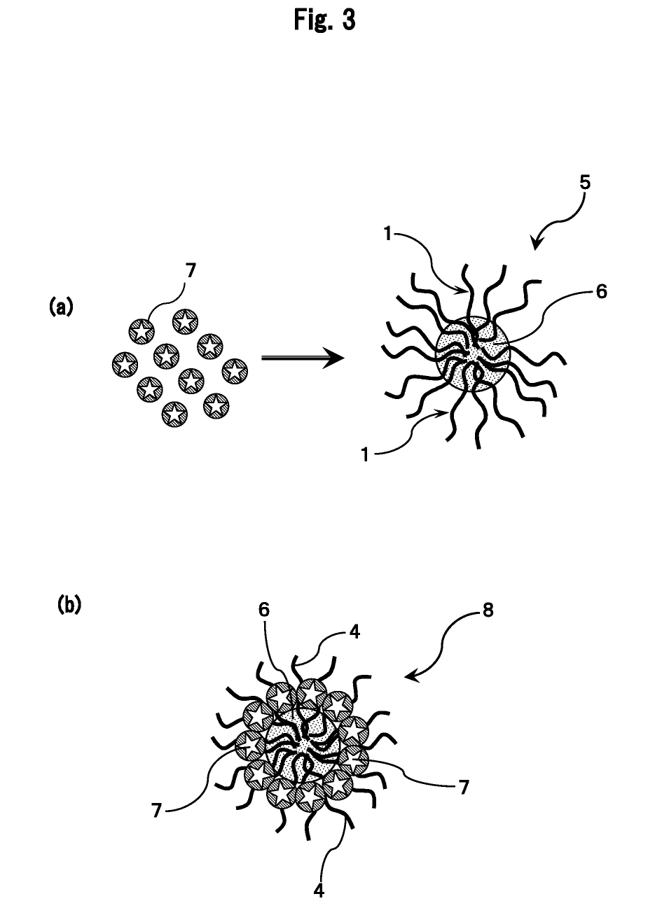

Subsequently, the present inventor allowed a nucleic acid 6 to interact with the block copolymer 1, so as to obtain a micelle structure (a nucleic acid polyplex 5) as shown in FIG. 2. In this nucleic acid polyplex 5, the nucleic acid 6 electrostatically binds to the block portion 2 in the polymer 1 to form a core portion. Other portions (the block portions 3 and 4, etc.) in the polymer 1 extend outward, so as to form a shell portion (an outer shell portion). Thereafter, as shown in FIG. 3(a), an anionic photosensitizing substance 7 was allowed to act on the nucleic acid polyplex 5.

As a result, as shown in FIG. 3(b), there was constructed a polymer micelle complex (a polyion complex 8), wherein the photosensitizing substance 7 was incorporated into (or embedded in) the shell portion of the nucleic acid polyplex 5.

In this polyion complex 8, the photosensitizing substance 7 is further coated with a polymer portion containing the block portion 4 (the PEG chain) of the block copolymer 1, and thus a complex with excellent structural stability can be obtained.

### 2. NUCLEIC ACID POLYPLEX

The nucleic acid polyplex of the present invention is characterized in that it comprises a specific cationic polymer and a nucleic acid. The present nucleic acid polyplex is a micelle complex, wherein the nucleic acid forms a core portion and the polymer forms a shell portion.

### (1) Cationic Polymer

A specific cationic polymer acting as a constituent of the nucleic acid polyplex of the present invention has a structure as a block copolymer represented by the following general formula (1):

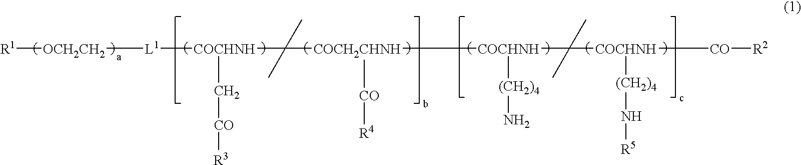

In the structural formula as shown in general formula (1), the symbol “-/-” representing a binding portion indicates that the ratio of the numbers of monomer units described at the left and right sides thereof and the sequence order are arbitrarily determined. For example, when monomer units “-A-” and “-B-” constituting a block portion are represented by “-A-/-B-” using the aforementioned symbol regarding a binding portion, it means that the ratio of the numbers of the constitutional units A and B is not limited, and also that the alignment sequence of individual A and B that are ligated to one another is not limited (they must be ligated to one another linearly, however). Accordingly, the number of either A or B may be 0, or A and B may be polymerized via either block polymerization or random polymerization. The total number of A and B is within the range of a polymerization degree determined for a block portion constituted with A and B (repeating unit numbers; “b” and “c” in general formula (1), for example).

In general formula (1), each of R¹ and R² that are terminal portions of the polymer independently represents a hydrogen atom or a substitutable linear or branched alkyl group containing 1 to 12 carbon atoms.

Examples of the aforementioned linear or branched alkyl group containing 1 to 12 carbon atom include a methyl group, an ethyl group, an n-propyl group, an isopropyl group, an n-butyl group, a sec-butyl group, a tert-butyl group, an n-pentyl group, an n-hexyl group, a decyl group, and an undecyl group.

Examples of a substituent of the aforementioned alkyl group include an acetalized formyl group, a cyano group, a formyl group, a carboxyl group, an amino group, an alkoxycarbonyl group containing 1 to 6 carbon atoms, an acylamide group containing 2 to 7 carbon atoms, a siloxy group, a silylamino group, and a trialkylsiloxy group (each alkylsiloxy group independently contains 1 to 6 carbon atoms).

When the aforementioned substituent is an acetalized formyl group, it is hydrolyzed under acidic, moderate conditions, so as to convert it to another substituent, a formyl group (aldehyde group; —CHO). In addition, when the aforementioned substituent (in particular, a substituent in R¹) is a formyl group, a carboxyl group, or an amino group, antibodies, the fragments thereof, or other functional proteins or proteins with target directivity may be bind to the alkyl group via the aforementioned groups.

In general formula (1), each of R³ and R⁴ independently represents a residue derived from an amine compound having a primary amine. A preferred example of the —R³ group and/or —R⁴ group is a group represented by the following general formula (2):

—[NH—(CH₂)ₘ₁]ₘ₂—X¹ (2)

[wherein X¹ represents a primary, secondary or tertiary amine compound, or an amine compound residue derived from a quaternary ammonium salt; and m1 and m2 are independent from each other and are also independent among the [NH—(CH₂)ₘ₁] units, and m1 represents an integer between 1 and 5 (preferably 2 or 3) and m2 represents an integer between 1 and 5 (preferably 2 to 5, and more preferably 2)].

In general formula (2), preferred examples of the —X¹ group (an amine compound residue) at the terminus include —NH₂, —NH—CH₃, —N(CH₃)₂, and groups represented by the following formulae (i) to (viii). In formula (vi), below, Y is a hydrogen atom, an alkyl group (containing 1 to 6 carbon atoms), or an aminoalkyl group (containing 1 to 6 carbon atoms), for example.

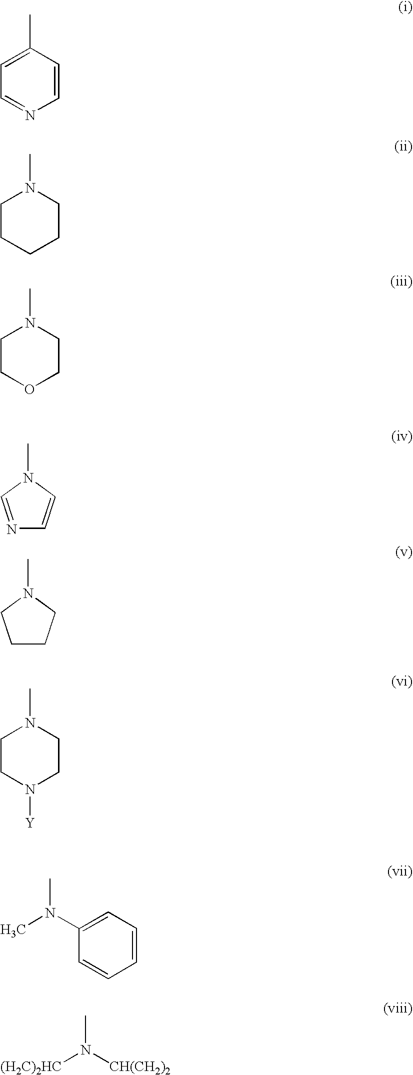

In general formula (1), R⁵ represents a residue containing a thiol group (—SH) or a residue containing a substituent of the thiol group. Preferred examples of such —R⁵ group include a residue represented by the following general formula (3):

—CO—(CH₂)ₙ—SH (3)

[wherein n represents an integer between 1 and 5 (preferably 2 or 3)]; and a residue represented by the following general formula (4):

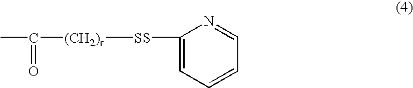

[wherein r represents an integer between 1 and 5 (preferably 2 or 3)].

In general formula (1), L¹ acting as a linker portion represents NH, CO, a group represented by the following general formula (5):

—(CH₂)ₚ₁—NH— (5)

[wherein p1 represents an integer between 1 and 5 (preferably 2 or 3)], or a group represented by the following general formula (6):

-L^{2a}-(CH₂)_{q1}-L^{3a}- (6)

[wherein L^{2a} represents OCO, OCONH, NHCO, NHCOO, NHCONH, CONH, or COO; and L^{3a} represents NH or CO; and q1 represents an integer between 1 and 5 (preferably 2 or 3)].

In general formula (1), each of a, b and c indicates the number of repeating units (polymerization degree) of each bloc portion.

Specifically, a indicates an integer between 100 and 500 (preferably 200 to 300).

In addition, b indicates an integer between 5 and 100 (preferably 20 to 50).

Moreover, c indicates an integer between 20 and 100 (preferably 40 to 80). Among others, the number of monomer units having a side chain containing —R⁵ is not limited, but it is preferably 1 to 20 in total, and more preferably 1 to 10 in total.

As stated above, the polymer represented by general formula (1) is considered to be a block copolymer having the following three block portions as constituents.

A block portion consisting of a polyethylene glycol (PEG) chain (a block portion with a polymerization degree of a)

A block portion having a side chain electrostatically binding to an anionic photosensitizing substance (a block portion with a polymerization degree of b having —R³ and/or —R⁴ at the side chain thereof)

A block portion having a side chain electrostatically binding to a nucleic acid (a block portion with a polymerization degree of c having —NH₂ and/or —NH— at the side chain thereof)

When the block portion with a polymerization degree of c comprises a side chain containing a —R⁵ group (a residue containing a thiol group or a substituent thereof), a reaction occurs between the polymers represented by general formula (1), and a cross-linked structure can be formed. By such crosslinking, the structure of the shell portion is stabilized, and the complex as a whole becomes further excellent in terms of structural stability.

The molecular weight (MW) of the polymer represented by general formula (1) is not limited, but it is preferably between 5,000 and 50,000, and more preferably between 10,000 and 30,000.

A method for producing the polymer represented by general formula (1) is not limited. Examples of such a production method include: a method comprising previously synthesizing a segment (a PEG segment) containing a block portion of PEG chain and a —R¹ group, polymerizing predetermined monomers to one terminus of the PEG segment (the terminus opposite to the —R¹ group) in a predetermined order, and then substituting or converting the side chain thereof, as necessary; and a method comprising previously synthesizing the aforementioned PEG segment and a block portion having a predetermined side chain and then ligating these components to each other. Various methods and conditions for various types of reactions used in the production methods can be selected or determined in accordance with ordinary methods.

The aforementioned PEG segment can be prepared by the method for producing a PEG segment portion of a block copolymer described in WO96/32434, WO96/33233, and WO97/06202, for example. The terminus opposite to the —R¹ group of the PEG segment is a portion corresponding to L¹ in general formula (1). Preferred examples of such a terminus opposite to the —R¹ group include —NH₂, —COOH, a group represented by the following general formula (7):

—(CH₂)ₚ₂—NH₂ (7)

[wherein p2 represents an integer between 1 and 5 (preferably 2 or 3)], and a group represented by general formula (8):

-L^{2b}-(CH₂)_{q2}-L^{3b} (8)

[wherein L^{2b} represents OCO, OCONH, NHCO, NHCOO, NHCONH, CONH, or COO; L^{3b} represents NH₂ or COOH; and q2 represents an integer between 1 and 5 (preferably 2 or 3)].

An example of a specific method for producing the polymer represented by general formula (1) is a method, which comprises: polymerizing N-carboxylic anhydride (NCA) of protected amino acid, such as β-benzyl-L-aspartate and Nε-Z-L-lysine, to the amino acid terminus of a PEG segment derivative having an amino acid group at the terminus thereof, so as to synthesize a block copolymer; and then substituting or converting the side chain of each block portion such that it becomes a side chain having the aforementioned desired properties.

### (2) Nucleic Acid

In the nucleic acid polyplex of the present invention, the type of a nucleic acid acting as a constituent of a core portion is not limited. Examples of such a nucleic acid include various types of DNA, RNA, and PNA (peptide nucleic acid), which can be used in gene therapy or the like. Preferred examples include plasmid DNA, antisense oligo DNA, and siRNA.

Since a core portion formed by aggregation of nucleic acid molecules becomes a polyanion, it is able to bind to the side chain of a certain block portion of the aforementioned cationic polymer by electrostatic interaction.

In the present invention, the core portion may comprise various substances whose functions are expressed in a cell, such as a physiologically active protein and various types of peptides, in addition to the aforementioned nucleic acid, as necessary.

Moreover, in another aspect of the present invention, a high-molecular-weight or low-molecular-weight “anionic substance” can be used as a constituent of the core portion. Examples of such an anionic substance include: high-molecular-weight substances such as a peptide hormone, a protein, an enzyme and a nucleic acid (DNA, RNA, or PNA); and low-molecular-weight substances (water-soluble compounds) having a charged functional group in a molecule thereof. This anionic substance does not include the after-mentioned anionic photosensitizing substance. On the other hand, this anionic substance includes a substance capable of changing the charged state of molecules having multiple functional groups in different charged states (anionic groups and cationic groups) to an anionic state by changing pH. Such anionic substances may be used singly or in combination of two or more types. It is not limited.

### (3) Nucleic Acid Polyplex

A nucleic acid polyplex is a core-shell-type micelle complex, wherein a nucleic acid interacts with a portion of a cationic polymer (a portion having a side chain electrostatically binding to the nucleic acid) to form a core portion, and wherein another portion of the aforementioned cationic polymer (a portion containing a block portion having a side chain electrostatically binding to an anionic photosensitizing substance and a block portion consisting of a PEG chain) forms a shell portion around the aforementioned core portion (see FIG. 2). In the present invention, the polymer represented by the aforementioned general formula (1) is used as a cationic polymer.

The nucleic acid polyplex of the present invention can be easily prepared by mixing a nucleic acid with a cationic polymer in a buffer, for example.

The mixing ratio between a cationic polymer and a nucleic acid is not limited. For example, the ratio (N/P ratio) between the total number (N) of amino groups in such a cationic polymer and the total number (P) of phosphoric acid groups in such a nucleic acid is preferably 0.5 to 5, and more preferably 1 to 2. The N/P ratio that is within the above range is preferable in that free polymers do not exist. The aforementioned amino groups in a cationic polymer mean terminal amino groups (—NH₂) of the side chain of a block portion with a polymerization degree of “d” in general formula (1). These are groups capable of electrostatically interacting with phosphoric acid groups in the nucleic acid so as to form an ionic bond.

The size of the nucleic acid polyplex of the present invention is not limited. For example, a particle size is preferably between 50 and 300 nm, and more preferably between 50 and 200 nm, according to the dynamic light scattering.

The nucleic acid polyplex of the present invention can be used as a constituent of the after-mentioned polyion complex of the present invention. In addition, in some cases, by the combined use of the nucleic acid polyplex with various types of known photosensitizing substances, the nucleic acid polyplex can be used as a device for delivering a nucleic acid into a target cell via endosome.

### 3. POLYION COMPLEX

The polyion complex of the present invention is a ternary (nucleic acid/anionic photosensitizing substance/cationic polymer) polymer micelle complex, which is characterized in that it comprises the aforementioned nucleic acid polyplex and an anionic photosensitizing substance.

Moreover, in another aspect, the polyion complex of the present invention also includes a ternary (anionic substance/anionic photosensitizing substance/cationic polymer) polymer micelle complex, which comprises a polyplex formed by using an anionic substance as a constituent of a core portion of the aforementioned nucleic acid polyplex, and an anionic photosensitizing substance.

### (1) Anionic Photosensitizing Substance

The type of an anionic photosensitizing substance used as a constituent of the polyion complex of the present invention is not limited. Various types of known anionic photosensitizing substances can be used. Such a photosensitizing substance may be excited by light with any type of wavelength region, such as ultraviolet light, visible light, and infrared radiation. A photosensitizing substance reactive with ultraviolet light or visible light, the light source of which is inexpensive and which is easy in handling, is preferable.

As an anionic photosensitizing substance, an anionic dendrimeric photosensitizing substance is preferable. In particular, as such an anionic dendrimer, a dendrimer having a metalloporphyrin ring is preferable, and a dendrimer containing metallophthalocyanine is more preferable (for example, a dendrimer represented by general formula (c) as described later). The term “metalloporphyrin ring” is used herein to mean a ring structure represented by the following general formula (a):

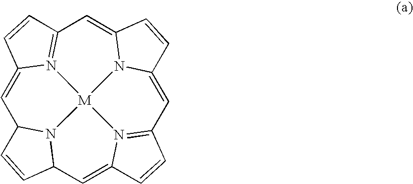

(wherein M represents a metal atom).

With regard to the aforementioned metalloporphyrin, the excited state and the oxidation state of oxygen are different depending on the type of a metal atom M acting as a central metal. As such a metal atom M, a metal capable of generating single oxygen while forming a stable metalloporphyrin ring-containing compound in a living body is preferable. Preferred examples of such a metal atom M include various types of metal atoms such as Zn, Mg, Fe, Cu, Co, Ni, and Mn. Among them, Zn, which has high energy in a photoexcited state and is advantageous in generation of single oxygen, is particularly preferable (the same holds for the metal atom M in general formulae (e), (f), and (g) as described later).

Preferred examples of an anionic dendrimeric photosensitizing substance include those represented by the following formulae (b) to (d):

q(−)PM (b)

q(−)PcM (c)

q(−)NcM (d)

[wherein, in formulae (b) to (d), q represents the number of charged atoms on the outer surface of a dendrimer; (−) represents the type of charge (namely, a negative charge); PM in formula (b), PcM in formula (c), and NcM in formula (d) represent dendrimers represented by the following general formulae (e), (f), and (g), respectively].

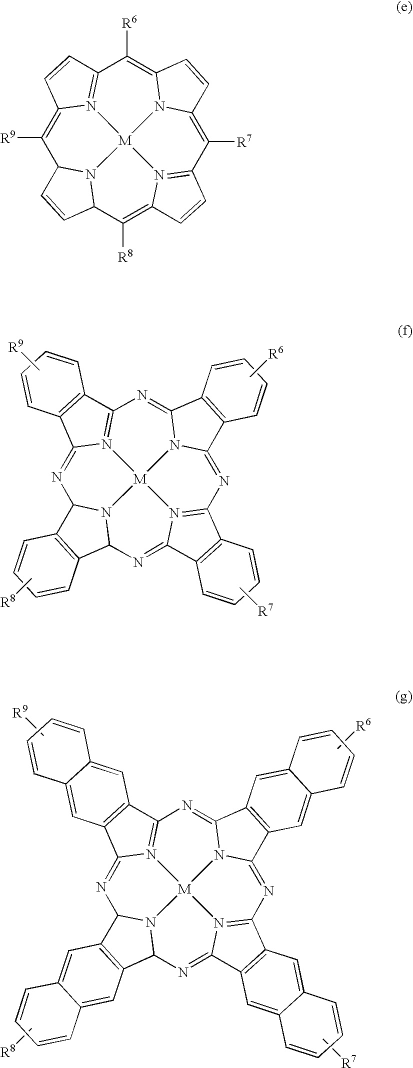

In the above general formulae (e), (f), and (g), M represents a metal atom; and each of R⁶, R⁷, R⁸ and R⁹ independently represents an anionic substituent or a dendron subunit containing an anionic substituent.

Herein, the type of an anionic substituent is not limited. Preferred examples of such an anionic substituent include acid anion groups such as a carboxylic acid group, a sulfonic acid group, and a phosphoric acid group.

A preferred example of a dendron subunit containing the aforementioned anionic substituent is a structure represented by the following general formula (h):

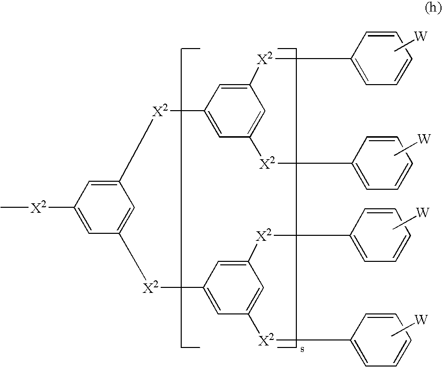

[wherein each X² independently represents a structural portion containing one or more oxygen atoms or carbon atoms (preferably —O—); s represents an integer between 1 and 25 (preferably 1 to 4); and each W independently represents one or multiple anionic substituents, or residues containing such substituents, and such W may bind to a benzene ring].

Herein, an anionic substituent in the dendron subunit is the same as that described above. A preferred example of a residue containing an anionic substituent is a residue having an anionic substituent at the terminus of a spacer molecular chain. A preferred example of such a spacer molecular chain is a hydrocarbon chain. Specifically, an alkyl chain is preferable, and an alkyl chain containing 25 or less carbon atoms is more preferable. Moreover, a molecular chain represented by the following general formula (j) is also preferable as a spacer molecular chain.

—C(X³)—X⁴R¹⁰—(CH₂R¹R²)ₜ— (d)

[wherein each of X³ and X⁴ independently represents one type selected from among an oxygen atom (O), a sulfur atom (S) and a nitrogen atom (N); R¹⁰ exists only in a case where X⁴ is N, and it represents a hydrocarbon group; R¹¹ and R¹² represent hydrocarbon groups or do not exist; and t represents an integer between 1 and 25 (preferably 1 to 6)].

When each of R¹⁰, R¹¹ and R¹² is a hydrocarbon group, the number of carbon atoms is preferably 25 or less, and more preferably 10 or less.

The aforementioned anionic dendrimer can be synthesized by known production methods, namely, a Divergent method involving the synthesis of a dendrimer from the center thereof towards the outer side (terminal portion) (D. A. Tomalia, et al., Polymer J., 17, 117 (1985)) or a Convergent method involving the synthesis of a dendrimer from the outer side thereof towards the center thereof (C. Hawker, et al., J. Chem. Soc. Chem. Commun., 1010 (1990)). For example, as a method for producing an anionic phthalocyanine dendrimer (DPc) represented by the aforementioned formula (6), a 3,5-dihydromethylphenol derivative acting as a monomer of the dendrimer is first allowed to react with isophthalate having a phenol hydroxyl group, and the protected phenol hydroxyl group is then deprotected. The aforementioned monomer reaction is repeated to obtain a dendrimer portion. Thereafter, phthalonitrile acting as a core of the dendrimer is introduced therein, and an oxidation-reduction reaction is then carried out in the presence of a metal (M), so as to obtain an anionic phthalocyanine dendrimer.

### (2) Polyion Complex

The polyion complex of the present invention is a core-shell-type ternary polymer micelle complex, wherein a core portion is coated with multiple anionic photosensitizing substances contained in the shell portion of the aforementioned nucleic acid polyplex of the present invention and wherein a portion containing a PEG chain of the aforementioned shell portion exists outside the photosensitizing substances (please see FIG. 3(b)). As described above, a portion of the cationic polymer represented by general formula (1) is used for the aforementioned shell portion in the present invention. This portion contains a portion (side chain) electrostatically interacting with the anionic photosensitizing substances. Accordingly, as a result of such interaction, a ternary polymer micelle complex consisting of the “nucleic acid/anionic photosensitizing substance/cationic polymer” is formed.

The polyion complex of the present invention can be easily prepared by mixing the aforementioned nucleic acid polyplex with the anionic photosensitizing substances in a buffer, for example.

The mixing ratio between the nucleic acid polyplex and the anionic photosensitizing substances is not limited. For example, the ratio (A/C; hereinafter referred to as “r ratio”) between the total number (A) of anionic groups in a photosensitizer and the total number (C) of cationic groups in the segment 3 of FIG. 1 is preferably 0.1 to 10, and more preferably 1 to 3. In particular, when the anionic photosensitizing substance is the aforementioned dendrimer-type photosensitizing substance, the r ratio is preferably 1 to 5, and more preferably 1 to 3. The r ratio that is within the aforementioned range is preferable in that free photosensitizers do not exist.

The size of the polyion complex of the present invention is not limited. For example, a particle size is preferably between 50 and 300 nm, and more preferably between 50 and 200 nm, according to the dynamic light scattering.

The polyion complex of the present invention can be preferably used as a device for delivering a nucleic acid into a target cell via endosome.

### 4. NUCLEIC ACID-DELIVERING DEVICE

The present invention provides a device for delivering a nucleic acid into a cell, which comprises the aforementioned polyion complex (a ternary polymer micelle complex). The nucleic acid-delivering device of the present invention can be used as a means for selectively and efficiently introducing a desired nucleic acid contained in the core portion of the polyion complex into a target cell via endosome, utilizing the principle of photodynamic therapy.

Specifically, a solution that contains a polyion complex including a desired nucleic acid is administered to a test animal, so that the polyion complex can be introduced into the endosome of various types of cells in the body. Thereafter, a target cell (a target tissue), into which the nucleic acid is to be introduced, is irradiated with light. In the cell irradiated with light, endosome-selective light disturbance occurs by the action of a photosensitizing substance contained in the polyion complex. Thereby, the nucleic acid is released from the endosome, and it is then introduced into the cytoplasm only in the target cell.

The nucleic acid-delivering device of the present invention can be applied to various types of animals such as a human, a mouse, a rat, a rabbit, a swine, a dog, and a cat, and thus the target animals are not limited. As an administration method to test animals, parenteral administration such as intravenous drip infusion is generally adopted. Various conditions such as a dose, the number of doses and an administration period can be determined, as appropriate, depending on the type of a test animal and the condition thereof. For light irradiation to a target cell, various types of light sources such as ultraviolet light (wavelength of 400 nm or less), visible light (wavelength between 400 and 700 nm), and infrared ray (wavelength of 700 nm or more) can be used. Light irradiation energy can also be determined, as appropriate. In addition, taking into consideration the influence upon cytotoxicity, the light irradiation time is preferably determined to be 0.1 to 60 minutes (more preferably 1 to 30 minutes), but it is not limited thereto.

The nucleic acid-delivering device of the present invention can be used in a therapy for introducing a desired nucleic acid into a cell that causes various types of diseases (gene therapy). Accordingly, the present invention can also provide a pharmaceutical composition comprising the aforementioned polyion complex, and a method for treating various types of diseases (in particular, gene therapy), using the aforementioned polyion complex (a nucleic acid-delivering device). It is to be noted that methods and conditions applied for administration and light irradiation are the same as those described above.

The aforementioned pharmaceutical composition can be prepared according to a common method by selecting and using, as appropriate, agents that are commonly used in drug manufacturing, such as an excipient, a filler, an extender, a binder, a wetting agent, a disintegrator, a lubricant, a surfactant, a dispersant, a buffer, a preservative, a solubilizer, an antiseptic, correctives, a soothing agent, a stabilizer, and an isotonizing agent. As the form of such a pharmaceutical composition, intravenous injection (including drops) is generally adopted. For example, the pharmaceutical composition of the present invention is provided in the form of a single dose ampule or a multidose container.

The aforementioned pharmaceutical composition and therapeutic method are effectively applied to, in particular, cancer from among various types of diseases.

### 5. NUCLEIC ACID-DELIVERING KIT

The nucleic acid-delivering kit of the present invention is characterized in that it comprises the aforementioned cationic polymer and anionic photosensitizing substance. This kit can be preferably used in gene therapy for various types of target cells such as cancer cells, etc.

In the kit of the present invention, the preservation state of the cationic polymer and anionic photosensitizing substance is not particularly limited. Taking into consideration their stability (preservative quality), usability, etc., these substances can be preserved in any given form such as a solution or powders.

The kit of the present invention may comprise other constituents, as well as the aforementioned cationic polymer and anionic photosensitizing substance. Examples of such other constituents include, but are not limited to, various types of buffers, various types of nucleic acids (plasmid DNA, antisense oligo DNA, siRNA, etc.), a buffer used for dissolution, and instruction for use (manual for use).

The kit of the present invention is used to prepare a polyion complex comprising, as a core portion, a desired nucleic acid to be introduced into a target cell. The prepared polyion complex can be effectively used as a device for delivering a nucleic acid into a target cell via endosome.

The present invention will be more specifically described in the following examples. However, these examples are not intended to limit the scope of the present invention.

### EXAMPLE 1

### Preparation of Nucleic Acid Polyplex

### (1) Synthesis of Cationic Block Copolymer

A cationic block copolymer was synthesized according to the following reaction formula (A). Specifically, first, polyethylene glycol having an amino group at one terminus was used as an initiator, and 40-fold molar amount of β-benzyl-L-aspartate-N-carboxy anhydride (BLA-NCA) was subjected to ring-opening polymerization in a mixed solvent of dimethylformamide (DMF)/dichloromethane at 30° C. Forty-eight hours later, a polymer solution was added dropwise to an excessive amount of diethyl ether, followed by the recovery by filtration with a filter. Thereafter, the resultant was washed with ether, and it was then recovered by filtration, so as to obtain PEG-b-PBLA in the form of white powders. The structure of the obtained polymer was confirmed by ¹H-NMR measurement and gel permeation chromatography (GPC) measurement. Subsequently, the synthesized diblock copolymer was dissolved in DMF, and ε-benzyloxycarbonyl(Z)-L-lysine N-carboxy anhydride (Lys(Z)-NCA) was further polymerized from the terminal amino group of the PBLA portion (40° C., 48 hours), followed by recovery by ether reprecipitation. At the same time, the terminal amino group of the polymer was acetylated by treating with acetic anhydride. The structure of the obtained polymer (PEG-b-PBLA-b-Lys(Z)) was confirmed by the ¹H-NMR measurement and the GPC measurement (molecular weight distribution M_{w}/Mₙ: 1.18).

400 mg of the thus obtained PEG-b-PBLA-b-Lys(Z) was dissolved in 8 mL of DMF, and 4-(3-aminopropyl)morpholine in a molar amount 10 times larger than the BLA residue was added thereto. The mixture was then reacted at 40° C. for 24 hours. The obtained reaction solution was recovered by ether reprecipitation, and it was then dissolved in trifluoroacetic acid. 30% HBr/acetic acid was added to the solution, and the obtained mixture was then stirred for 1 hour, thereby deprotecting the Z group. Thereafter, the resultant was reprecipitated in diethyl ether, and it was then dialyzed to 0.01 N HCl, followed by freeze-drying, so as to obtain PEG-b-PMPA-b-PLL in the form of white powders (311 mg).

Subsequently, a thiol group was introduced into the PLL chain to stabilize the crosslinking of the DNA contained therein. In such a thiol group-introducing reaction, each of PEG-b-PMPA-b-PLL and SPDP was dissolved in N-methyl-2-pyrrolidone, to which 5% by weight of LiCl had been added, and the mixture was then reacted for 24 hours, followed by the recovery by ether reprecipitation.

With regard to the polymerization degree of each block portion of the obtained block copolymer, a=272, b=36, and c=50. The molecular weight (Mw) was 30,200. In addition, by the reaction of SPDP, thiol groups (-SS-Py) were introduced into 17 out of 50 residues of PLL.

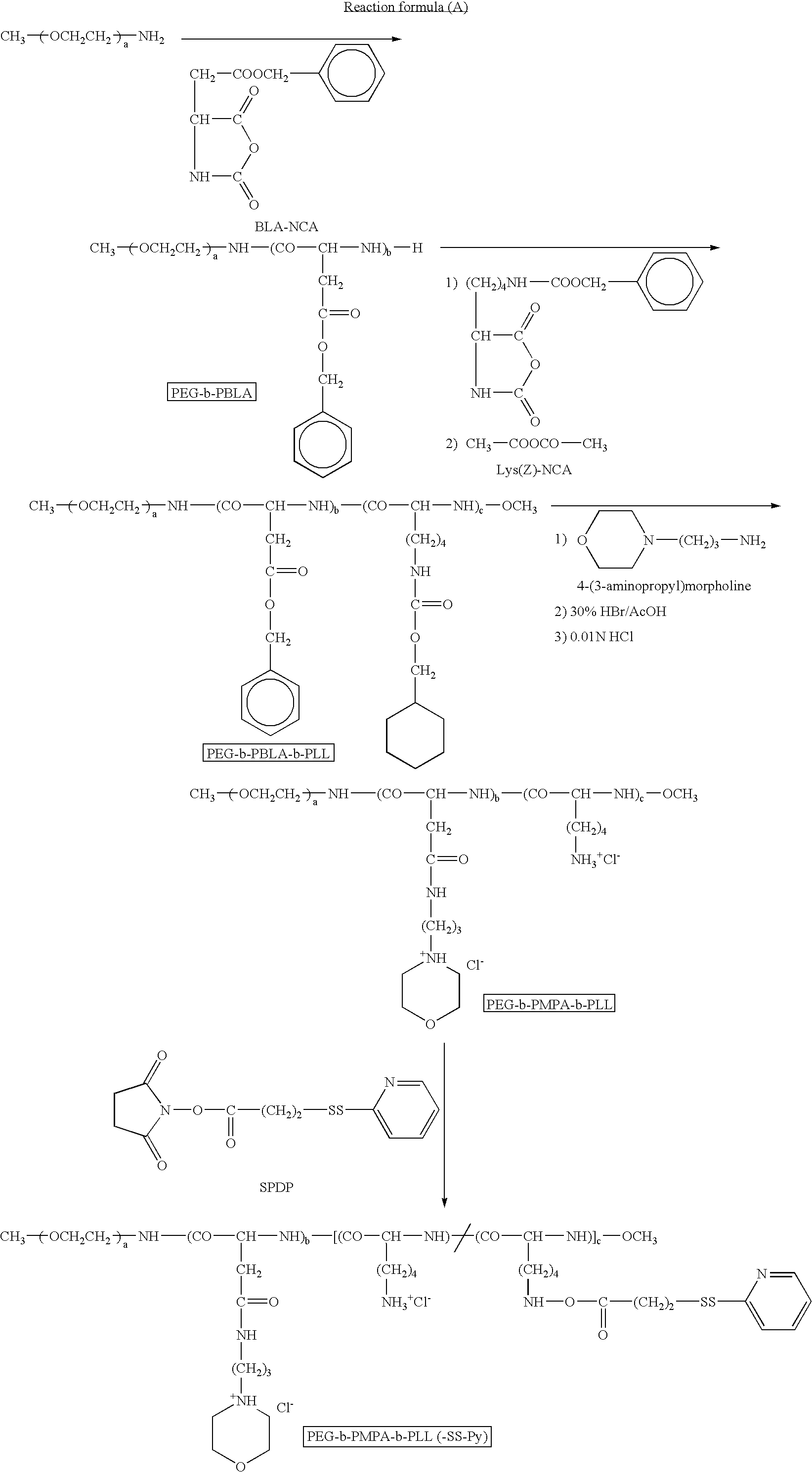

### (2) Used Nucleic Acid

As a nucleic acid to be delivered into a cell, a luciferase expression plasmid acting as a reporter gene (hereinafter referred to as “pDNA”) was used.

### (3) Preparation of Nucleic Acid Polyplex

A cationic block copolymer that had been pre-treated with 10 mg a reducer dithiothreitol (DTT) was mixed with pDNA in a 10 mM Tris buffer (pH 7.4), so as to prepare a nucleic acid polyplex containing the pDNA in the core portion thereof. The mixing ratio (the amino groups (N) in the polymer/the phosphoric acid groups (P) in the pDNA; N/P ratio) was determined to be 2. A protecting group -SS-Py and the reducer DTT were eliminated. Further, in order to form a disulfide bond between PLL chains in the inner core of the nucleic acid polyplex, the reaction solution was dialyzed to a 10 mM Tris buffer solution (pH 7.4) containing 2% dimethyl sulfoxide (DMSO) as an oxidizer for 72 hours, using a dialysis membrane with a molecular weight cut off of 1,000.

The particle size of the prepared nucleic acid polyplex was found to be 106 nm according to the dynamic light scattering.

### EXAMPLE 2

### Preparation of Polyion Complex

(1) Synthesis of dendrimer-type anionic photosensitizing substance

A dendron subunit was synthesized according to reaction formula (B1) set forth below, and a dendrimer was then synthesized according to reaction formula (B2) set forth below.

Specifically, in reaction formula (B1), dimethyl-5-hydroxyphthalate was first protected by t-butyldiphenylsilyl chloride, and lithium ammonium hydride was then used for reduction of the compound. Thereafter, the resultant was allowed to react with dimethyl-5-hydroxyphthalate as a monomer, and the t-butyldiphenylsilyl group used as a protecting group was eliminated. Further, the obtained compound was allowed to react with a compound protected and reduced in the same manner as described above. This reaction is carried out repeatedly, so as to synthesize a dendron subunit.

In reaction formula (B2), nitrophthalonitrile was allowed to bind to the dendron subunit in the presence of a base. Using pentanol as a solvent, zinc acetate was added to the reaction product, and the mixture was refluxed, so as to synthesize a dendrimer.

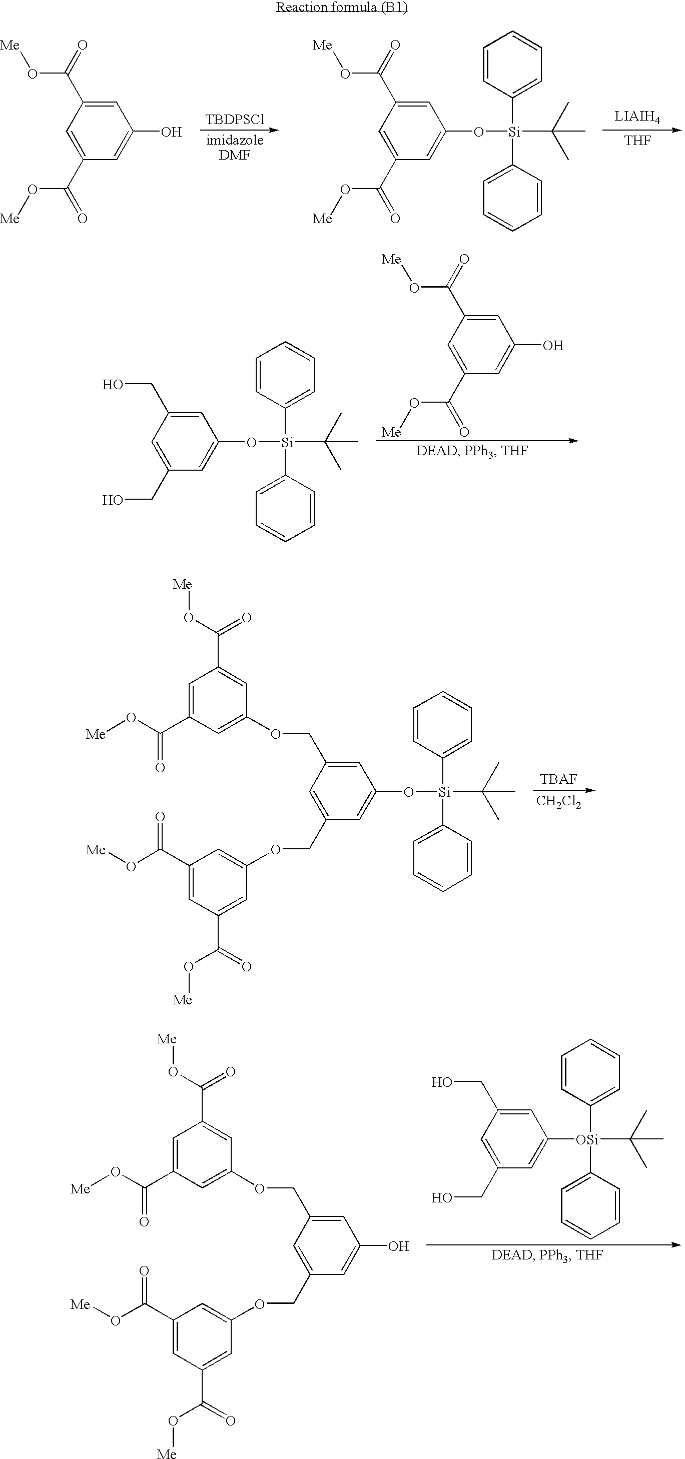

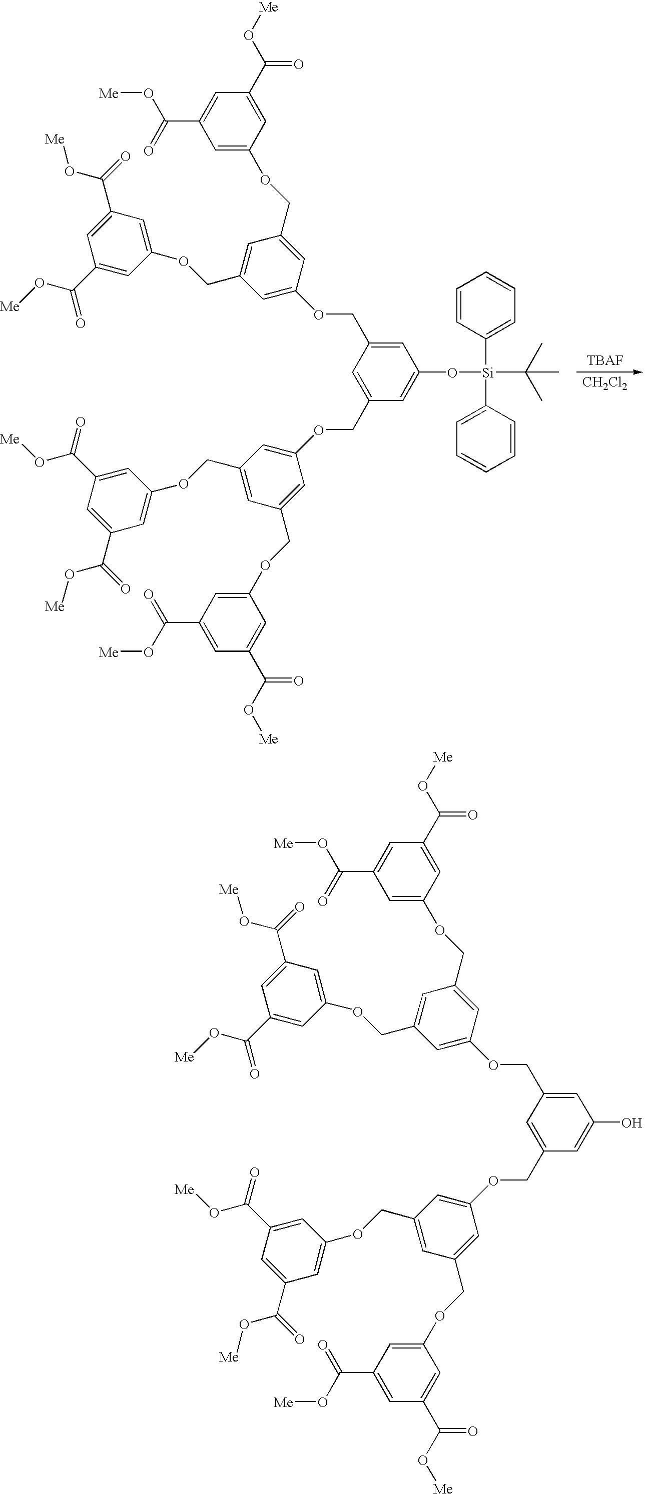

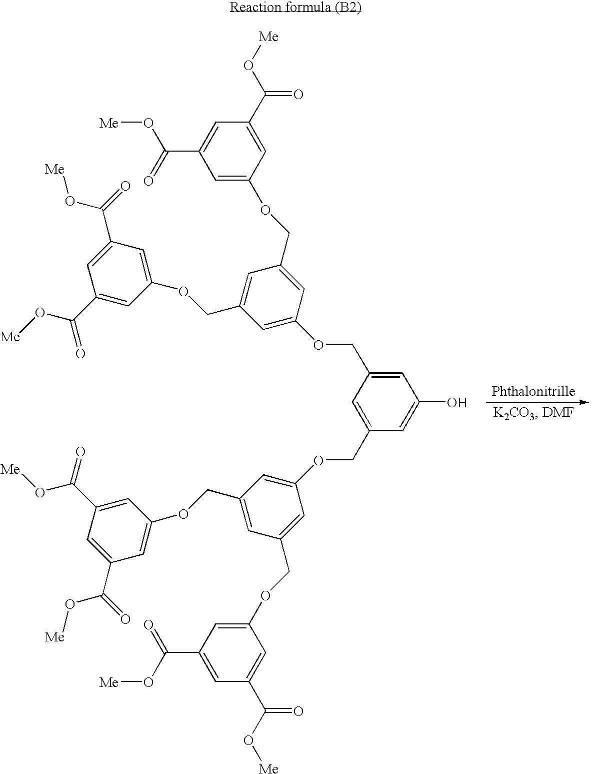

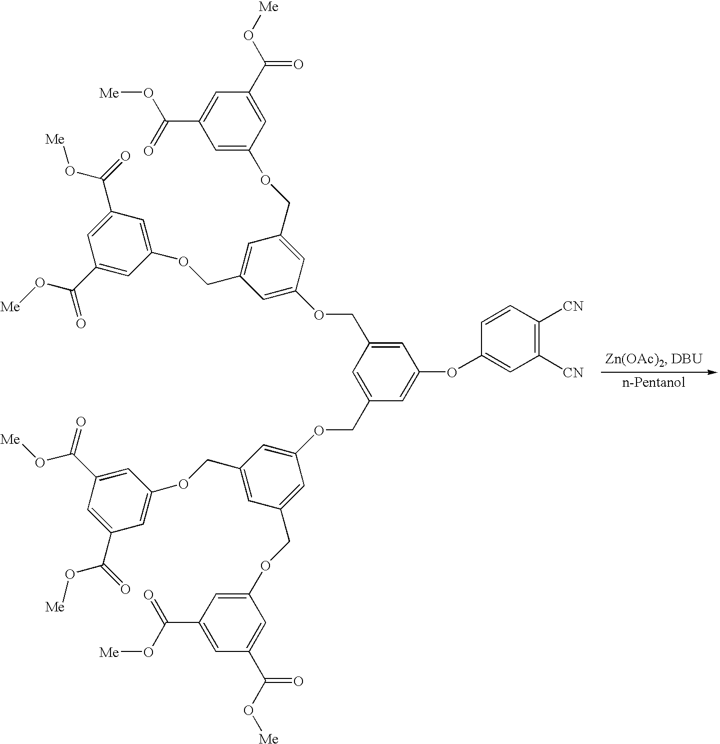

Furthermore, the dendrimer obtained from reaction formula (B) was treated with an aqueous NaOH solution, so that the functional group at the terminus of each dendron subunit was converted to a carboxylic acid group, thereby obtaining an anionic phthalocyanine dendrimer ([32(−)(L3)₄PcZn]) as shown below.

The obtained phthalocyanine dendrimer was dissolved in Na₂HPO₄, resulting in a concentration of 10 mM. A small amount of NaOH was then added to the solution, so that the dendrimer was completely dissolved therein.

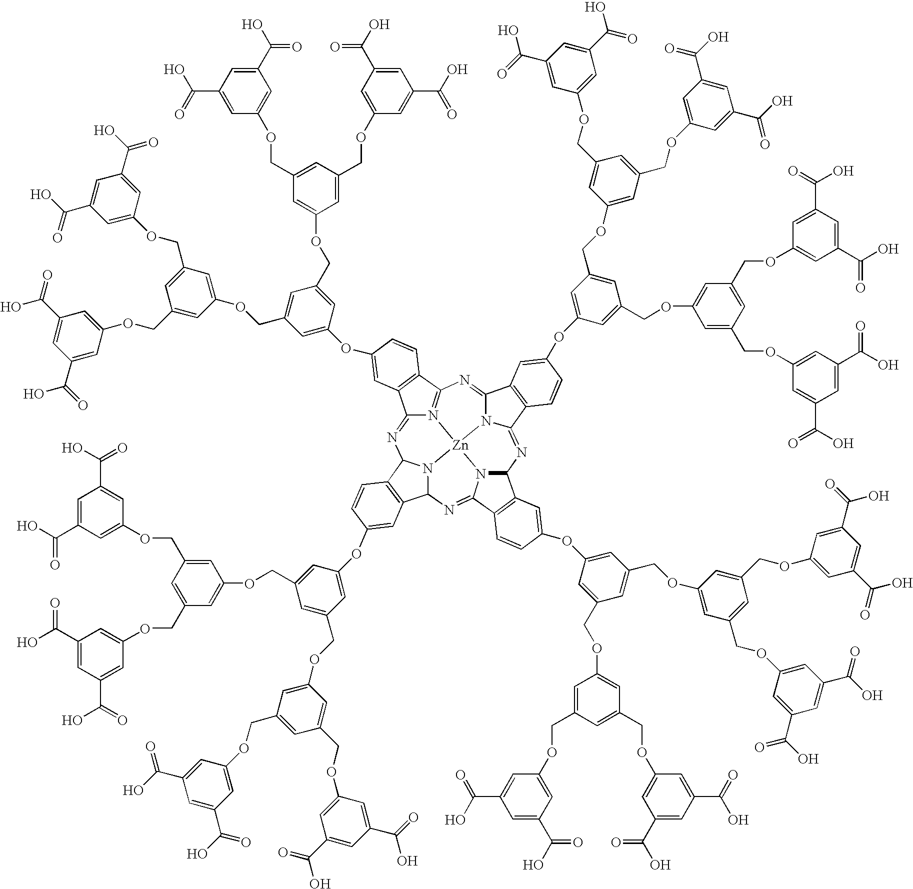

### (2) Preparation of Polyion Complex

A solution containing the nucleic acid polyplex obtained in Example 1 was mixed with a solution of the aforementioned anionic phthalocyanine dendrimer (DPc), so as to obtain a solution containing a ternary polymer micelle complex (polyion complex) consisting of pDNA/anionic phthalocyanine dendrimer/cationic block copolymer.

As such polyion complexes, polyion complexes having the mixing ratios (the total number of anionic groups in a photosensitizer/the total number of cationic groups in a PMPA chain; r ratio) as shown in the following Table 1 were prepared, individually. The particle size and dispersion degree of such a polyion complex were measured according to the dynamic light scattering. As a result, it was found that the polyion complexes having mixing ratios of 1 to 3 (in particular, 2 and 3) were preferable in terms of particle size and dispersion degree.

| Mixing ratio | Particle size | |
|---|---|---|
| (r ratio) | (nm) | Dispersion degree |
| 0 | 105 | 0.190 |
| 1 | 90.6 | 0.089 |
| 2 | 104 | 0.071 |
| 3 | 95.2 | 0.062 |
| 4 | 137 | 0.262 |
| 5 | 101 | 0.263 |

### EXAMPLE 3

### Absorbance Spectrum Measurement

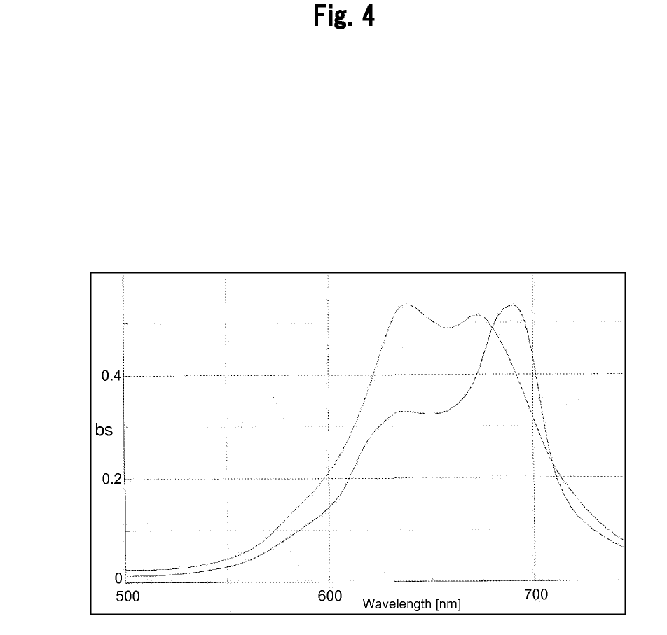

FIG. 4 shows the visible light absorbance spectrum derived from an anionic phthalocyanine dendrimer (DPc) of the polyion complex prepared in Example 2 (r=1; amount relative to DNA: 100 μg/ml in 10 mM PBS).

As a result, it was confirmed that absorption around 680 nm of DPc was decreased and absorption around 630 nm was increased because of the presence of the nucleic acid polyplex. This phenomenon occurred because DPc formed a complex with the nucleic acid polyplex, and these results demonstrate that a polyion complex was formed.

### EXAMPLE 4

### Measurement of Zeta Potential

The zeta potentials of the polyion complexes having different r ratios (r=0 to 5) prepared in Example 2 were measured using a Zetasizer (Sysmex Corporation).

Consequently, as shown in Table 5, it was confirmed that, as the r ratio increased, the zeta potential was changed from a slightly positively charged state to a negatively charged state.

In a state where DPc was not added (r=0), a free cationic polymer layer was present in the intermediate layer of the nucleic acid polyplex, and thus it was positively charged (the absolute value was low because the shell portion was coated with a PEG layer). However, by addition of DPc having a negative charge (an increase in the r ratio), the DPc interacts with the intermediate layer, and thus it is considered that it has a negative zeta potential.

### EXAMPLE 5

### Light Irradiation Intensity and Cytotoxicity

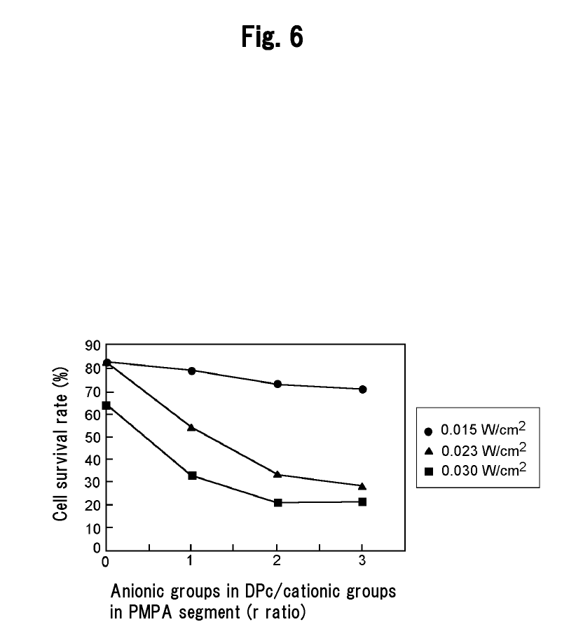

10,000 human hepatoma Huh-7 cells were inoculated on a 24-well multiplate, and the cells were then cultured for 24 hours in a DMEM medium, to which 10% fetal bovine serum had been added. Thereafter, each of the polyion complexes prepared in Example 2 having different r ratios (r=0, 1, 2 and 3; amount relative to DNA: 1 μg) was added to the resulting cells, and the mixture was further cultured for 6 hours. Thereafter, washing with a phosphate buffer and the exchange of the medium were conducted, and light irradiation (wavelength: 400 to 700 nm) was then carried out while changing the intensity of the light applied. After completion of the light irradiation, the cells were further cultured for 48 hours. Thereafter, the survival rate of the cells was evaluated by MTT assay. The results are shown in FIG. 6.

### EXAMPLE 6

### Light Irradiation Time and Gene Expression Level

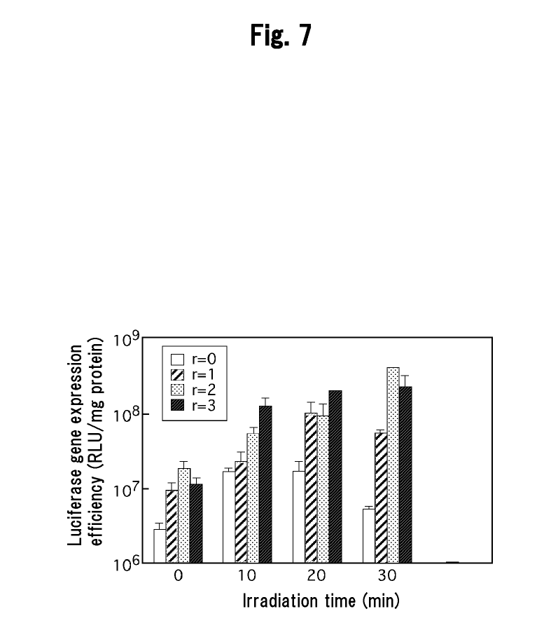

10,000 human hepatoma Huh-7 cells were inoculated on a 24-well multiplate, and the cells were then cultured for 24 hours in a DMEM medium, to which 10% fetal bovine serum had been added. Thereafter, each of the polyion complexes prepared in Example 2 having different r ratios (r=0, 1, 2 and 3; amount relative to DNA: 1 μg) was added to the resulting cells, and the mixture was further cultured for 6 hours. Thereafter, washing with a phosphate buffer and the exchange of the medium were conducted, and the cells were then irradiated with light (wavelength: 400 to 700 nm), using light of 0.030 W/cm2 having halogen lamp of 300 W as a light source. After completion of the light irradiation, the cells were further cultured for 48 hours. Thereafter, the gene expression efficiency was evaluated by luciferase assay. The gene expression level was obtained in the form of Relative Light Unit (RLU)/mg of protein amount. The results are shown in FIG. 7.

As shown in FIG. 7, in the case of a polyion complex of r=2, it was confirmed that the gene expression efficiency was increased to 50 times or more the original efficiency by light irradiation for 30 minutes. In addition, in the case of polyion complexes of other r ratios (r=1 or 3) as well, the same above tendency was confirmed regarding an increase in the gene expression efficiency by light irradiation. Thus, using a polyion complex, light-selective and efficient gene transfer could be achieved.

### EXAMPLE 7

### Light Irradiation Time and Cytotoxicity

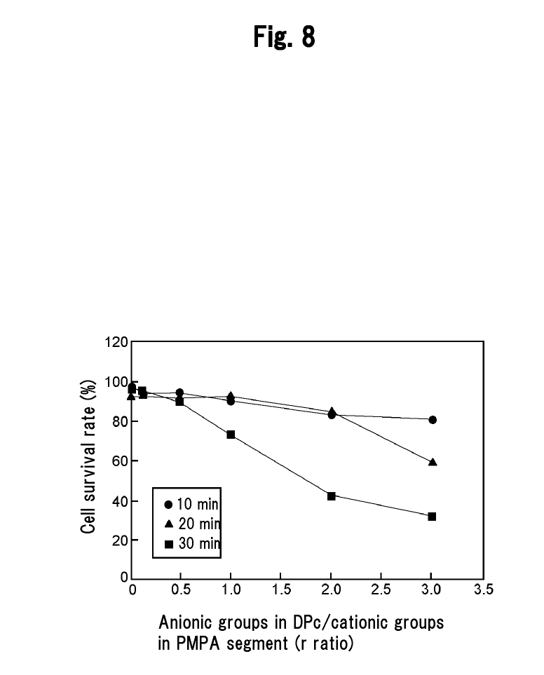

The survival rate of cells was evaluated by the MTT assay under the same experimental conditions as those in Example 6. The results are shown in FIG. 8.

It was confirmed that the cell survival rate was decreased to 30% to 40% under conditions where polyion complexes of r=2 and 3, which exhibit the highest gene expression efficiency in FIG. 7, were used, and where light was applied for 30 minutes. It was found that significant phototoxicity was observed while efficient gene expression was achieved under the aforementioned conditions. However, under conditions where polyion complexes of r=1 and 2 were used and where light was applied for 20 minutes, although gene expression efficiency was increased by an order of magnitude or more by light irradiation (FIG. 7), a significant decrease in the cell survival rate as shown in FIG. 8 was not observed. From these results, it was confirmed that the polyion complex of the present invention is able to achieve photoselective and efficient gene transfer without provoking phototoxicity.

### INDUSTRIAL APPLICABILITY

The present invention provides a polyion complex that is extremely excellent in terms of ability to retain a photosensitizing substance in serum and is able to exhibit extremely high structural stability, and a nucleic acid polyplex used as a constituent of the polyion complex.

The polyion complex of the present invention enables efficient and selective introduction of a nucleic acid into a target cell, and because of its high structural stability in serum, it also enables an effective delivery of a nucleic acid via intravenous administration. Thus, the present polyion complex is extremely excellent in terms of practical application and usability.

Moreover, since the polyion complex of the present invention comprises a polymer chain (a portion of a cationic polymer) containing PEG on the surface thereof, it is excellent in terms of bio-compatibility, and it is able to reduce the interaction with an ionic protein in blood to the minimum. From this respect as well, structural stability in serum is increased.

Furthermore, the present invention also provides a device for delivering a nucleic acid into a cell using the aforementioned polyion complex, and a kit for delivering a nucleic acid into a cell, which comprises a constituent of the aforementioned polyion complex (a cationic polymer, an anionic photosensitizing substance).

1. A nucleic acid polyplex, which comprises a cationic polymer represented by the following general formula (1) and a nucleic acid: 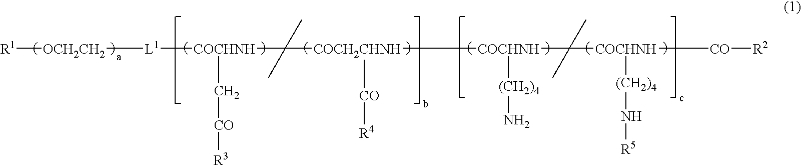
[wherein each of R¹ and R² independently represents a hydrogen atom or a substitutable linear or branched alkyl group containing 1 to 12 carbon atoms;
each of R³ and R⁴ independently represents a residue derived from an amine compound having a primary amine;
R⁵ represents a residue containing a thiol group or a substituent thereof;
L¹ represents NH, CO, a group represented by the following general formula (5):
—(CH₂)ₚ₁—NH— (5)
(wherein p1 represents an integer between 1 and 5), or a group represented by the following general formula (6):
-L^{2a}-(CH₂)_{q1}-L^{3a}- (6)
(wherein L^{2a} represents OCO, OCONH, NHCO, NHCOO, NHCONH, CONH or COO, L^{3a} represents NH or CO, and q1 represents an integer between 1 and 5);
a represents an integer between 100 and 500; b represents an integer between 5 and 100; c represents an integer between 20 and 100; and
the symbol “/” indicates that the ratio of the numbers and the sequence order of monomer units described at the left and right sides thereof are arbitrarily determined].

2. The nucleic acid polyplex according to claim 1, wherein the —R³ group and/or —R⁴ group in the polymer is a group represented by the following general formula (2):
—[NH—(CH₂)ₘ₁]ₘ₂—X¹ (2)
(wherein X¹ represents a primary, secondary or tertiary amine compound, or an amine compound residue derived from a quaternary ammonium salt; and m1 and m2 are independent from each other and are also independent among the [NH—(CH₂)ₘ₁] units, and m1 represents an integer between 1 and 5 and m2 represents an integer between 1 and 5).

3. The nucleic acid polyplex according to claim 1, wherein the —NH₂ group in the polymer and the nucleic acid bind to each other by electrostatic interaction.

4. The nucleic acid polyplex according to claim 1, wherein the nucleic acid forms a core portion and the polymer forms a shell portion.

5. A polyion complex, which comprises the nucleic acid polyplex according to claim 1 and an anionic photosensitizing substance.

6. The polyion complex according to claim 5, wherein the photosensitizing substance is a dendrimer.

7. The polyion complex according to claim 6, wherein the dendrimer has a metalloporphyrin ring.

8. The polyion complex according to claim 5, wherein the —R³ group and/or —R⁴ group in the polymer and the photosensitizing substance bind to each other by electrostatic interaction.

9. The polyion complex according to claim 5, wherein the nucleic acid forms a core portion as a result of being coated with the photosensitizing substance, and the polymer forms a shell portion.

10. The polyion complex according to claim 9, wherein the shell portion is formed by a portion comprising at least a polyethylene glycol chain of the polymer.

11. A device for delivering a nucleic acid into a cell, which comprises the polyion complex according to claim 5.

12. A kit for delivering a nucleic acid into a cell, which comprises a cationic polymer represented by the following general formula (1) and an anionic photosensitizing substance: 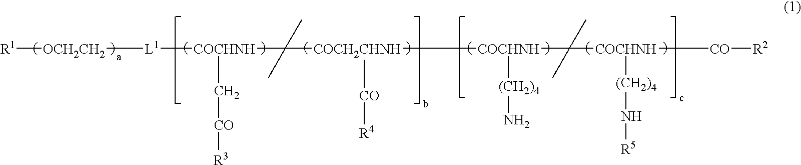
[wherein each of R¹ and R² independently represents a hydrogen atom or a substitutable linear or branched alkyl group containing 1 to 12 carbon atoms;
each of R³ and R⁴ independently represents a residue derived from an amine compound having a primary amine;
R⁵ represents a residue containing a thiol group or a substituent thereof,
L¹ represents NH, CO, a group represented by the following general formula (5):
—(CH₂)ₚ₁—NH— (5)
(wherein p1 represents an integer between 1 and 5), or a group represented by the following general formula (6):
-L^{2a}-(CH₂)_{q1}-L^{3a}- (6)
(wherein L^{2a} represents OCO, OCONH, NHCO, NHCOO, NHCONH, CONH or COO, L^{3a} represents NH or CO, and q1 represents an integer between 1 and 5);
a represents an integer between 100 and 500; b represents an integer between 5 and 100; c represents an integer between 20 and 100; and
the symbol “/” indicates that the ratio of the numbers and the sequence order of monomer units described at the left and right sides thereof are arbitrarily determined].

13. A cationic polymer represented by the following general formula (1): 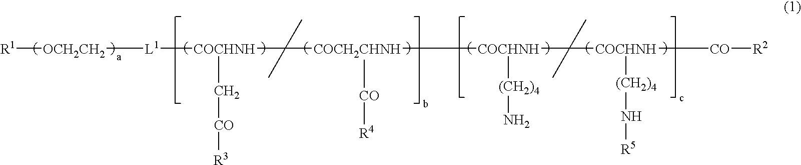
[wherein each of R¹ and R² independently represents a hydrogen atom or a substitutable linear or branched alkyl group containing 1 to 12 carbon atoms;
each of R³ and R⁴ independently represents a residue derived from an amine compound having a primary amine;
R⁵ represents a residue containing a thiol group or a substituent thereof;
L¹ represents NH, CO, a group represented by the following general formula (5):
—(CH₂)ₚ₁—NH— (5)
(wherein p1 represents an integer between 1 and 5), or a group represented by the following general formula (6):
-L^{2a}-(CH₂)_{q1}-L^{3a}- (6)
(wherein L^{2a} represents OCO, OCONH, NHCO, NHCOO, NHCONH, CONH or COO, L^{3a} represents NH or CO, and q1 represents an integer between 1 and 5);
a represents an integer between 100 and 500; b represents an integer between 5 and 100; c represents an integer between 20 and 100; and
the symbol “/” indicates that the ratio of the numbers and the sequence order of monomer units described at the left and right sides thereof are arbitrarily determined].
